(19) United States
(12) Patent Application Publication
(10) Pub. No.: US 2006/0024351 A1
(43) Pub. Date: Feb. 2, 2006

(54) Hydrating breath freshening beverage compositions and method therefor

(76) Inventor: Diane Bradford, Las Vegas, NV (US)

Correspondence Address: WEISS & MOY PC, 4204 NORTH BROWN AVENUE, SCOTTSDALE, AZ, 85251, US

(21) Appl. No.: 10/900,435

(22) Filed: Jul. 28, 2004

(51) Int. Cl. A61K 47/00; A61K 36/534

(52) U.S. Cl. 424/439; 424/747; 424/778

(57) ABSTRACT

A hydrating beverage comprises an effective amount of at least one breath freshening additive in a suitable amount of water to hydrate a person to prevent halitosis and bad breath when the hydrating beverage is consumed by the person and distributed to the mouth, the throat and the stomach of the person for a selected period of time as determined by the person. The at least one breath freshening additive is selected from the group consisting of menthol, thymol, eucalyptol, methyl salicylate, vanillin, peppermint oil, spearmint oil, and clove oil. The hydrating beverage comprises about 95-99.99 percent by weight of water and about 0.01-5 percent by weight of a combination of at least one of the breath freshening additive, at least one sweetener, at least one anti-microbial agent, at least one preservative and at least one surfactant.

### FIELD OF THE INVENTION

This invention relates generally to breath freshening compositions and, more specifically, to hydrating breath freshening beverage compositions and methods of combining both hydration of a person and providing breath freshening by consuming hydrating breath freshening beverage compositions.

### BACKGROUND OF THE INVENTION

As health consciousness has generally increased throughout the world, it has been common to see people consuming bottled waters of various kinds. In a warm climate such as Las Vegas, Nev. people regularly carry water during the driest times of the year because of the low humidity climate.

At the same time, it is also common for people to freshen their breath by consuming various gums and confections, examples of which include well-known brands such as CERTS™, ALTOIDS™, ORBIT™, POCKETPAKS™, TIC TAC™, and so on. Also well-known are mouth washes which require a person to expectorate the mouth wash rather than swallowing it. These products all are effective for a limited time only and require timely consumption in situations that may not be conducive to regular use. For example, chewing gums is often considered to be socially unacceptable in many situations, while using a mouth wash is usually reserved for home consumption. None of these breath freshening products combine the features of both hydration and breath freshening.

Recently, Tasker Capital Corp. announced that they will be marketing and distributing a beverage called BREATH-REPHRESH™, which allegedly aids in the prevention of cavities and plaque accumulation. No benefit is provided for other potential sources of bad breath such as the stomach of a person. In addition, the hydration of a person is also not a disclosed benefit of using this product. According to Tasker Capital Corp. this product consists of water, xylitol, Shitake mushroom extract, vitamin C, vitamin B6, citric acid, glucose, fructose, sorbit, butyl p-hydroxy benzoate, propylene glycol, sodium benzoate, ginger and copper sulfate (no amounts disclosed). Tasker Capital Corp. disclosed that the product will be introduced in the fall of 2004.

The current inventor of this disclosure has recognized the need for a combined hydrating and breath freshening beverage. The disclosure of this application addresses improved compositions and a method to provide hydrating breath freshening beverages, which overcome the limitations of the confections that are currently available and also are an improvement over the product that may be introduced into the marketplace as discussed above.

### SUMMARY OF THE INVENTION

It is an object of the present disclosure to provide improved hydrating breath freshening compositions in the form of a consumable beverage suitable for hydrating and orally freshening a person.

It is a further object of the present disclosure to provide improved hydrating breath freshening compositions in the form of a consumable beverage suitable for hydrating and orally freshening a person over an extended period of time.

It is a still further object of the present disclosure to provide improved hydrating breath freshening compositions in the form of a consumable beverage in bottled form thereby facilitating use of the beverage.

It is a further object of the present disclosure to provide a method to combine both hydration of a person and provide breath freshening by consuming a hydrating and breath freshening beverage composition.

### BRIEF DESCRIPTION OF THE PREFERRED EMBODIMENTS

In accordance with one embodiment of the present invention, a hydrating breath freshening beverage is disclosed. The hydrating beverage comprises, in combination, a beverage comprising a suitable amount of water to hydrate a person and further comprising an effective amount of at least one breath freshening additive in the water thereby preventing halitosis and bad breath when the hydrating beverage is consumed by the person and distributed to the mouth, the throat and the stomach of the person for a selected period of time as determined by the person. The at least one breath freshening additive is selected from the group consisting of menthol, thymol, eucalyptol, methyl salicylate, vanillin, peppermint oil, spearmint oil, and clove oil, and the hydrating beverage comprises about 95-99.99 percent by weight of the water and about 0.01-5 percent by weight of a combination of at least one of the breath freshening additive, at least one sweetener, at least one anti-microbial agent, at least one preservative and at least one surfactant.

In accordance with a second embodiment of the present embodiment, a hydrating breath freshening beverage is disclosed. The hydrating breath freshening beverage comprises, in combination, a beverage comprising a suitable amount of water to hydrate a person and further comprising an effective amount of at least one breath freshening additive in the water thereby preventing halitosis and bad breath when the hydrating beverage is consumed by the person and distributed to the mouth, the throat and the stomach of the person for a selected period of time as determined by the person. The water and the at least one breath freshening additive are bottled thereby facilitating use by the person for an extended period of time.

In accordance with a third embodiment of the present embodiment, a method of combining hydration of a person and providing a breath freshening beverage is disclosed. The method comprises the steps of, providing a beverage comprising a suitable amount of water to hydrate a person; providing the beverage further comprising an effective amount of at least one breath freshening additive in the water; and preventing halitosis and bad breath when the hydrating beverage is consumed by the person and distributed to the mouth, the throat and the stomach of the person for a selected period of time as determined by the person.

The foregoing and other objects, features, and advantages of the invention will be apparent from the following, more detailed description of the preferred embodiments of the invention.

### DETAILED DESCRIPTION OF THE PREFERRED EMBODIMENTS

According to the disclosure, a hydrating breath freshening beverage comprises a beverage comprising a suitable amount of water to hydrate a person and further comprises an effective amount of at least one breath freshening additive in water thereby preventing halitosis and bad breath when the hydrating beverage is consumed by the person and distributed to the mouth, the throat and the stomach of the person for a selected period of time as determined by the person. The combination of water and the at least one breath freshening additive are bottled thereby facilitating use by the person for an extended period of time. It is understood that the term water as used in the disclosure applies to the various grades of water that are available, such as tap water, distilled water, mineral water, carbonated water and the like. The hydrating beverage comprises about 95-99.99 percent by weight of water and about 0.01-5 percent by weight (total weight of additives in water) of a combination of at least one anti-microbial agent, at least one surfactant, at least one sweetener and at least one breath freshening additive. A person drinking the hydrating breath freshening beverage may sip portions of the beverage throughout a day, or alternatively may consume several bottles of the beverage according to the person's need to be hydrated and/or freshen the person's breath after events such as eating, smoking and the like.

The at least one breath freshening additive is selected from the group consisting of menthol, thymol, eucalyptol, methyl salicylate, vanillin, peppermint oil, spearmint oil, and clove oil. It is understood that various other breath freshening additives suitable for prolonged human consumption may be effectively used. In one embodiment, the at least one breath freshening additive comprises about 0.001-20 percent by weight of the total weight of additives in water of the hydrating beverage composition. In a second more preferred embodiment, the at least one breath freshening additive comprises about 0.1-20 percent by weight of the total weight of additives in water of the hydrating beverage composition. In a third most preferred embodiment, the at least one breath freshening additive comprises about 10-15 percent by weight of the total weight of additives in water of the hydrating beverage composition.

The hydrating breath freshening beverage composition may also comprise at least one sweetener selected from the group consisting of aspartame, saccharin, potassium acesulfame, sucralose, D-Tagatose, mannitol and sorbitol. It is understood that natural sweeteners such as sucrose, fructose, honey and the like may also be used in the hydrating breath freshening beverage composition, although the above described (i.e., aspartame, saccharin, etc.) substantially non-tooth decaying, low carbohydrate content sweeteners are preferred. In a preferred embodiment, the at least one sweetener comprises about 0.005-1.5 percent by weight of the total weight of additives in water of the hydrating beverage composition.

It is believed that some portion of halitosis may be caused by sulfur containing compounds generated by bacteria in the oral cavity or in the remainder of the digestive system. Consequently, the hydrating breath freshening beverage composition may further comprise at least one anti-microbial agent selected from the group consisting of copper salts of citric acid, zinc salts of citric acid, copper salts of gluconic acid and zinc salts of gluconic acid thereby preventing further halitosis-causing bacterial growth. Zn and Cu salts of these organic acids are believed to be food safe. It is understood that any Zn and Cu salt that is food safe and binds to a bacterial cell wall of the kind described above would be suitable for use in the hydrating breath freshening beverage composition. In a preferred embodiment, the at least one anti-microbial agent comprises about 0.15-1.5 percent by weight of the total weight of additives in water of the hydrating beverage composition.

The hydrating breath freshening beverage composition may further comprise at least one preservative selected from the group consisting of sodium benzoate and a substituted phenolic compound. These preservatives may both provide preservative characteristics to the beverage compositions and halitosis preventative characteristics in combination with the above-mentioned one anti-microbial agent. In a preferred embodiment, the at least one preservative comprises about 0.001-1 percent by weight of the total weight of additives in water of the hydrating beverage composition.

The hydrating breath freshening beverage may also be colored and therefore comprises at least one food-grade colorant. Examples of food grade colorants include FD & C yellow #5, yellow #10, FD & C green #3 and the like. Typically the at least one colorant comprises less than about 0.05 percent of the total weight of additives in water of the hydrating beverage composition. It is understood that various nutrients may also comprise a portion of the total weight of additives in water of the hydrating beverage composition.

At least one surfactant selected from the group consisting of polysorbates, glyceryl ester of a long chain fatty acid and glycerol may be added to the hydrating breath freshening beverage. It is understood that a mixture of surfactants that may be cationic, anionic and non-ionic may be used to facilitate dissolution of any non-dissolving additives of the above described hydrating beverage composition. Typically the at least one surfactant comprises less than about 8 percent of the total weight of additives in water of the hydrating beverage composition. Polysorbates are poly(oxyethylene)-poly(oxypopylene) block copolymers, and a typical polysorbate that may be used is Polysorbate 80 having a molecular weight of no more than about 12,000.

The container housing the hydrating breath freshening beverage may comprise a suitable plastic or glass. An example of a suitable plastic is polyester. The container may be sized for the convenience of a user. One convenient size of container may hold about 1 pint of water.

The present invention further comprises a method to combine both hydration of a person and provide breath freshening by consuming a hydrating breath freshening beverage composition. The method comprises the steps of providing a beverage comprising a suitable amount of water to hydrate a person; providing the beverage further comprising an effective amount of at least one breath freshening additive in the water; and preventing halitosis and bad breath when the hydrating beverage is consumed by the person and distributed to the mouth, the throat and the stomach of the person for a selected period of time as determined by the person.

The method further comprises the steps of bottling a combination of the water and the at least one breath freshening additive thereby facilitating use by the person for an extended period of time, and consuming the combination of the water and at least one breath freshening additive. The method further comprises the steps of providing the hydrating beverage comprises about 95-99.99 percent by weight of the water and about 0.01-5 percent by weight of a combination of at least one breath freshening additive, at least one sweetener, at least one anti-microbial agent, at least one preservative and at least one surfactant; providing the at least one breath freshening additive is selected from the group consisting of menthol, thymol, eucalyptol, methyl salicylate, vanillin, peppermint oil, spearmint oil, and clove oil; providing the at least one sweetener is selected from the group consisting of aspartame, saccharin, potassium acesulfame, sucralose, D-Tagatose, mannitol and sorbitol; providing the at least one anti-microbial agent is selected from the group consisting of copper salts of citric acid, zinc salts of citric acid, copper salts of gluconic acid and zinc salts of gluconic acid thereby preventing further halitosis-causing bacterial growth; providing the at least one preservative is selected from the group consisting of sodium benzoate and a substituted phenolic compound; and providing the at least one surfactant is selected from the group consisting of polysorbates, glyceryl ester of a long chain fatty acid and glycerol.

While the disclosure has been particularly shown and described with reference to preferred embodiments thereof, it will be understood by those skilled in the art that the foregoing and other changes in form and details may be made therein without departing from the spirit and scope of the disclosure.

What is claimed is:

1. A hydrating breath freshening beverage comprising, in combination:
a beverage comprising a suitable amount of water to hydrate a person and further comprising an effective amount of at least one breath freshening additive in said water thereby preventing halitosis and bad breath when said hydrating beverage is consumed by the person and distributed to the mouth, the throat and the stomach of the person for a selected period of time as determined by the person;
said at least one breath freshening additive selected from the group consisting of menthol, thymol, eucalyptol, methyl salicylate, vanillin, peppermint oil, spearmint oil, and clove oil; and
said hydrating beverage comprising about 95-99.99 percent by weight of said water and about 0.01-5 percent by weight of a combination of at least one of said breath freshening additive, at least one sweetener, at least one anti-microbial agent, at least one preservative and at least one surfactant.

2. The hydrating beverage according to claim 1 wherein said water and said at least one breath freshening additive are bottled thereby facilitating use by the person for an extended period of time.

3. A hydrating breath freshening beverage comprising, in combination: a beverage comprising a suitable amount of water to hydrate a person and further comprising an effective amount of at least one breath freshening additive in said water thereby preventing halitosis and bad breath when said hydrating beverage is consumed by the person and distributed to the mouth, the throat and the stomach of the person for a selected period of time as determined by the person.

4. The hydrating beverage according to claim 3 wherein said water and said at least one breath freshening additive are bottled thereby facilitating use by the person for an extended period of time.

5. The hydrating beverage according to claim 3 further comprising said at least one breath freshening additive selected from the group consisting of menthol, thymol, eucalyptol, methyl salicylate, vanillin, peppermint oil, spearmint oil, and clove oil.

6. The hydrating beverage according to claim 3 wherein said hydrating beverage comprises about 95-99.99 percent by weight of said water and about 0.01-5 percent by weight of a combination of at least one breath freshening additive, at least one sweetener, at least one anti-microbial agent, at least one preservative and at least one surfactant.

7. The hydrating beverage according to claim 3 further comprising at least one sweetener selected from the group consisting of aspartame, saccharin, potassium acesulfame, sucralose, D-Tagatose, mannitol and sorbitol.

8. The hydrating beverage according to claim 3 further comprising at least one anti-microbial agent selected from the group consisting of copper salts of citric acid, zinc salts of citric acid, copper salts of gluconic acid and zinc salts of gluconic acid thereby preventing further halitosis-causing bacterial growth.

9. The hydrating beverage according to claim 3 further comprising at least one preservative selected from the group consisting of sodium benzoate and a substituted phenolic compound.

10. The hydrating beverage according to claim 3 further comprising at least one food-grade colorant.

11. The hydrating beverage according to claim 3 further comprising at least one surfactant selected from the group consisting of polysorbates, glyceryl ester of a long chain fatty acid and glycerol.

12. The hydrating beverage according to claim 3 further comprising by percent weight of the total weight of a combination of additives in said water of said beverage, about 10-15 percent by weight of said breath freshening additive, about 0.005-1.5 percent by weight of at least one sweetener, about 0.15-1.5 percent by weight of at least one anti-microbial agent, about 0.001-1 percent by weight of at least one preservative, less than about 0.05 percent by weight of at least one food colorant, and less than about 8 percent by weight of at least one surfactant.

13. A method of combining hydration of a person and providing a breath freshening beverage comprising the steps of:
providing a beverage comprising a suitable amount of water to hydrate the person;
providing said beverage further comprising an effective amount of at least one breath freshening additive in said water; and
preventing halitosis and bad breath when said hydrating beverage is consumed by the person and distributed to the mouth, the throat and the stomach of the person for a selected period of time as determined by the person.

14. The method according to claim 13 further comprising the steps of:
bottling a combination of said water and said at least one breath freshening additive thereby facilitating use by the person for an extended period of time; and
consuming said combination of said water and at least one breath freshening additive.

15. The method according to claim 13 further comprising the steps of:
providing said hydrating beverage comprises about 95-99.99 percent by weight of said water and about 0.01-5 percent by weight of a combination of at least one breath freshening additive, at least one sweetener, at least one anti-microbial agent, at least one preservative and at least one surfactant;
providing said at least one breath freshening additive is selected from the group consisting of menthol, thymol, eucalyptol, methyl salicylate, vanillin, peppermint oil, spearmint oil, and clove oil;
providing said at least one sweetener is selected from the group consisting of aspartame, saccharin, potassium acesulfame, sucralose, D-Tagatose, mannitol and sorbitol;
providing said at least one anti-microbial agent is selected from the group consisting of copper salts of citric acid, zinc salts of citric acid, copper salts of gluconic acid and zinc salts of gluconic acid thereby preventing further halitosis-causing bacterial growth;
providing said at least one preservative is selected from the group consisting of sodium benzoate and a substituted phenolic compound; and
providing said at least one surfactant is selected from the group consisting of polysorbates, glyceryl ester of a long chain fatty acid and glycerol.
